(19) United States
(12) Patent Application Publication
(10) Pub. No.: US 2008/0025929 A1
(43) Pub. Date: Jan. 31, 2008

(54) Topical Formulations for the Treatment of Skin Conditions

(75) Inventors: Graham W. Burton, Ottawa (CA); Janusz Daroszweski, Ottawa (CA)

Correspondence Address: CLARK & ELBING LLP, 101 FEDERAL STREET, BOSTON, MA, 02110, US

(73) Assignee: Chemaphor, Inc., Ottawa (CA)

(21) Appl. No.: 10/589,700

(22) PCT Filed: Feb. 17, 2005
(86) PCT No.: PCT/IB05/01369

§ 371 Date: Aug. 10, 2007

Related U.S. Application Data
(60) Provisional application No. 60/546,043, filed on Feb. 19, 2004.

(51) Int. Cl. A61K 8/00; A61P 17/18

(52) U.S. Cl. 424/59

(57) ABSTRACT

The invention features compositions for topical administration of an oxidatively transformed carotenoid or 2-methyl-6-oxo-2,4-heptadienal and methods of treating skin conditions therewith.

### BACKGROUND OF THE INVENTION

The invention relates to the formulation of carotenoid oxidation products and their use in the treatment of skin conditions.

Carotenoids are naturally occurring substances which contain extensively conjugated polyene chains, which give rise to their many varied and brilliant colors. Carotenoids are reactive towards molecular oxygen (O₂). For example, beta-carotene has been shown to have antioxidant properties at the low oxygen pressures found in tissues and pro-oxidant properties at higher pressures (Burton and Ingold, Science, 224:569 (1984)). The oxidation of carotenoids with molecular oxygen has been shown to produce a mixture of polymeric material and many low molecular weight products, including 2-methyl-6-oxo-2,4-heptadienal (U.S. Pat. No. 5,475,006 and PCT Publication No. 96/05160).

A variety of biological activities have been attributed to carotenoids. For example, carotenoids have been shown to retard the development of some experimentally induced animal tumors (N. I. Krinsky, Annu. Rev. Nutr., 13, 561-587 (1993); Matthews-Roth, Curr. Top. Nutr. Dis., 22:17-38 (1989)). Furthermore, epidemiological evidence indicates that carotenoid intake correlates inversely with the incidence of some types of cancer (Peto et al, Nature, 290:201-208 (1981)). Beta-carotene and phytoene have been used in combination with UV light therapy to treat psoriasis (U.S. Pat. No. 4,642,318).

In contrast, little is known about the biological activity of the oxidation products of carotenoids or their use for the treatment of skin conditions.

There is a need for new compositions and methods for the treatment of skin conditions, such as photoaging, dandruff, psoriasis, eczema, keloids, keratosis, and warts, among others.

### SUMMARY OF THE INVENTION

In a first aspect, the invention features a composition formulated for topical administration including from 0.0001% to 5% (w/w) oxidatively transformed carotenoid.

In a second aspect, the invention features a composition formulated for topical administration including from 0.0001% to 5% (w/w) 2-methyl-6-oxo-2,4-heptadienal.

In one embodiment of the above aspects, the composition further includes an antioxidant. The antioxidant can be selected from thiols, sulphoximines, metal chelators, fatty acids, vitamins, phenols, stilbenes, uric acid, mannose, selenium and propyl gallate. Desirably, the antioxidant is vitamin E.

In another embodiment of the above aspects, the composition further includes one or more solubilizing excipients wherein the class of excipient is selected from the group consisting of polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, ionic surfactants, tocopherol esters, and sterol esters.

In yet another embodiment of the above aspects, the composition further includes a UV light blocker, a corticosteroid, an antihistamine, a retinoid, 5-fluorouracil, epinephrine, anthralin, calcipotriene, methotrexate, masprocol, trimethaxate gluconate, cyclosporin, paclitaxel, 5-amino levulinic acid, bergasol, benzoporphyrin, hydroxy acid, retinoic acid, diphencyprone, aldara, imiquimod, gamma-linolenic acid, chloroxine, coal tar, ketoconazole, pyrithione, salicylic acid, zinc salts, selenium sulfide, piroctone olamine, sulphur, or mixtures thereof. Exemplary UV light blockers include those selected from amino benzoic acids, benzophenones, camphors, cinnamates, dibenzoyl methanes, salicylates, metal oxides, and mixtures thereof. Exemplary antihistamines include mepyramine, diphenhydramine, and antazoline. Exemplary corticosteroids include alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, and triamcinolone acetonide.

In still another embodiment of the above aspects, the composition is formulated as a cream, lotion, spray, stick, ointment, soap, body wash, shampoo, or mask.

In a third aspect, the invention features a method of treating a skin condition in a mammal by applying oxidatively transformed carotenoid to the skin of the mammal in an amount sufficient to treat the skin condition.

In a fourth aspect, the invention features a method of treating a skin condition in a mammal by applying 2-methyl-6-oxo-2,4-heptadienal to the skin of the mammal in an amount sufficient to treat the skin condition.

In one embodiment of the above methods, the skin condition is dandruff. For the treatment of dandruff, the method can also include the administration of chloroxine, coal tar, ketoconazole, pyrithione, salicylic acid, zinc salts, selenium sulfide, piroctone olamine, sulphur, or combination thereof to the mammal.

In another embodiment of the above methods, the skin condition is psoriasis. For the treatment of psoriasis, the method can also include the administration of a corticosteroid, 5-fluorouracil, epinephrine, anthralin, calcipotriene, methotrexate, masprocol, trimethaxate gluconate, retinoids, cyclosporin, paclitaxel, 5-amino levulinic acid, bergasol, benzoporphyrin, or combination thereof to the mammal.

In yet another embodiment of the above methods, the skin condition is photoaging. For the treatment of photoaging, the method can also include the administration of a UV light blocker, hydroxy acid, retinoic acid, gamma-linolenic acid, or combination thereof to the mammal.

In still another embodiment of the above methods, the skin condition is eczema. For the treatment of eczema, the method can also include the administration of an antihistamine, corticosteroid, or combination thereof to the mammal. Corticosteroids useful for the treatment of eczema using the methods described herein include alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, and triamcinolone acetonide. Antihistamines useful for the treatment of eczema using the methods described herein include mepyramine, diphenhydramine, and antazoline.

In still another embodiment of the above methods, the skin condition is selected from warts, keloids, and keratosis.

In a fifth aspect, the invention features a container including a composition of the invention and packaged under an atmosphere purged of oxygen gas.

In a sixth aspect, the invention features a composition comprising from 0.001% to 3% by weight antioxidant and oxidatively transformed carotenoid or 2-methyl-6-oxo-2,4-heptadienal. Desirably, the amount of antioxidant included in the composition is from 0.01% to 1% (w/w), or even 0.05% to 0.5% (w/w), based on the total weight of the composition. Exemplary antioxidants include vitamin E, among others.

As used herein, the term “treating” refers to administering a composition formulated for topical application for prophylactic and/or therapeutic purposes. To “prevent” a disease or condition refers to prophylactic treatment of a patient who is not yet ill, but who is susceptible to, or otherwise at risk of, a particular disease or condition. To “treat” a disease or use for “therapeutic treatment” refers to administering treatment to a patient already suffering from a disease to improve the patient's condition. Thus, in the claims and embodiments, treating is the administration to an animal either for therapeutic or prophylactic purposes.

By “sufficient amount” is meant the amount of a compound required to treat or prevent a skin condition. The sufficient amount of oxidatively transformed carotenoid or 2-methyl-6-oxo-2,4-heptadienal used to practice the invention for therapeutic or prophylactic treatment of any particular condition varies depending upon the age, body weight, and general health of the subject and the condition to be treated. Ultimately, the attending physician or veterinarian will decide the appropriate amount and dosage regimen. Such amount is referred to as a “sufficient” amount.

By “mammal” is meant, without limitation, any mammal including a human, dog, cat, horse, or cow. Desirably, the mammal is a human.

As used herein, “carotenoid” refers to naturally-occurring pigments of the terpenoid group that can be found in plants, algae, bacteria, and certain animals, such as birds and shellfish. Carotenoids include carotenes, which are hydrocarbons (i.e., without oxygen), and their oxygenated derivatives, including the alcoholic forms known as xanthophylls. Examples of carotenoids include lycopene; beta-carotene; zeaxanthin; echinenone; isozeaxanthin; astaxanthin; canthaxanthin; lutein; citranaxanthin; β-apo-8′-carotenic acid ethyl ester; hydroxy carotenoids, such as alloxanthin, apocarotenol, astacene, astaxanthin, capsanthin, capsorubin, carotenediols, carotenetriols, carotenols, citranaxanthin, cryptoxanthin, decaprenoxanthin, denethylated-spheroidine, epilutein, fucoxanthin, hydroxycarotenones, hydroxyechinenones, hydroxylycopene, lutein, lycoxanthin, neurosporine, phytoene, phytofluoene, rodopin, spheroidine, torulene, violaxanthin, and zeaxanthin; and carboxylic carotenoids, such as apocarotenoic acid, β-apo-8′-carotenoic acid, azafrin, bixin, carboxylcarotenes, crocetin, diapocarotenoicacid, neurosporaxanthin, norbixin, and lycopenoicacid.

As used herein “oxidatively transformed carotenoid” refers to a carotenoid which has been reacted with up to 6 to 8 molar equivalents of oxygen resulting in a mixture of very low molecular weight oxidative cleavage products and a large proportion of oligomers and polymers. The resulting reaction produces a mixture that includes molecular species having molecular weights ranging from about 100 to 8,000 Daltons. The polymeric material is believed to be formed by the many possible chemical recombinations of the various oxidative fragments that are formed. Methods of making the oxidatively transformed carotenoid are described in U.S. Pat. No. 5,475,006 and U.S. Ser. No. 08/527,039, filed Sep. 12, 1995, each of which are incorporated herein by reference.

As used herein, “photoaging” is a term used to describe the changes in appearance and/or function of human skin as a result of repeated exposure to sunlight, and especially regarding wrinkles and other changes in the appearance of the skin. Photoaging in human skin is characterized clinically by coarseness, wrinkles, mottled pigmentation, sallowness, and laxity.

As used herein, “an atmosphere purged of oxygen gas” refers compositions of the invention packaged for storage or for sale wherein the packaged compositions are largely free of oxygen gas (e.g., less than 5%, desirably less than 1%, of the gas that is in contact with the composition is oxygen gas). This can be accomplished by, for example, replacing ambient air in a package with an inert atmosphere, such as nitrogen, argon, or neon, or by packaging the composition in a vacuum.

The compositions and methods of the invention can be used to treat skin conditions, such as dandruff, psoriasis, eczema, keloids, keratosis, and warts. Furthermore, the formulations can be used to treat the symptoms of photoaging of the skin, such as coarseness, wrinkles, mottled pigmentation, and sallowness.

Other features and advantages of the invention will be apparent from the following Detailed Description and the claims.

### DETAILED DESCRIPTION

The invention provides compositions for the topical administration of oxidatively transformed carotenoid and 2-methyl-6-oxo-2,4-heptadienal. The compositions are useful for the treatment of a variety of skin conditions.

### Topical Formulations

Examples of skin care products which may be prepared using the formulations of the invention include, without limitation, a skin cream, a facial cream; a cleanser, a toner, a day cream, a night cream, a day lotion, an eye cream, a facial mask (e.g., firming, moisturizing, purifying, deep-cleansing), an anti-aging cream, an anti-wrinkle cream, an anti-puffiness product, a cold weather cream, a foot cream; a facial scrub; a hand cream; hair care products; beauty treatment products; a perfume; a bath and body product; a suncare product; or combinations thereof.

Hair care products which may be prepared in accordance with the invention include, for example, a shampoo, a conditioner, a re-conditioner, a mousse, a gel, a hair spray, a hair mascara, a hot oil treatment product, a dye, a hair mask, a deep conditioning treatment product, a coloring product, a hair-repair product, a permanent wave product, or combinations of thereof.

Beauty treatment products which may be prepared in accordance with the invention include, without limitation, a waxing product, a pedicure product, a manicure product, a facial product, a beauty lift product, a massage product and a aroma-therapy product, and combinations thereof.

Perfumes which may be prepared in accordance with the invention include, without limitation, an eau de toilette, an eau de perfume, a perfumed bath, body lotion, shower gel, aftershave, and combinations thereof.

Bath and body products which may be prepared in accordance with the invention include for example a shower gel, including an exfoliating shower gel, a foaming bath product (e.g., gel, soap or lotion), a milk bath, a body wash, a soap (including liquid and bar soap), a cleanser, including a gel cleanser, a liquid cleanser, a cleansing bar, a body lotion, a body spray, mist or gel, an essential lotion, a slimming lotion, bath effervescent tablets, a hand and nail cream, a bath/shower gel, a shower cream, a cellulite smoothing product, a deodorant, a dusting powder, an antiperspirant, a depilatory cream, a shaving product (e.g., a shaving cream, a gel, a foams and an after-shave, after-shave moisturizer), and combinations thereof.

The oxidatively transformed carotenoid or 2-methyl-6-oxo-2,4-heptadienal can be included in a suncare product, such as a sunscreen; a sunblocker; an after sun lotion milks and gel; a bum lotion; a tanning lotion, spray and milk; a sunless self-tanning cream, spray and lotion; and combinations thereof.

Any conventional pharmacologically and cosmetically acceptable vehicles may be used. For example, the compounds may also be administered in liposomal formulations that allow compounds to enter the skin. Such liposomal formulations are described in U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; and PCT Publication No. WO 96/40061. Examples of other appropriate vehicles are described in U.S. Pat. No. 4,877,805 and EP Publication No. 0586106A1. Suitable vehicles of the invention may also include mineral oil, petrolatum, polydecene, stearic acid, isopropyl myristate, polyoxyl 40 stearate, stearyl alcohol, or vegetable oil.

The formulations can include various conventional colorants, fragrances, thickeners (e.g., xanthan gum), preservatives, humectants, emollients (e.g., hydrocarbon oils, waxes, or silicones), demulcents, solubilizing excipients, dispersants, penetration enhancers, plasticizing agents, preservatives, stabilizers, demulsifiers, wetting agents, sunscreens, emulsifiers, moisturizers, astringents, deodorants, and the like can be added to provide additional benefits and improve the feel and/or appearance of the topical preparation.

The formulations are typically used for the prophylaxis and/or treatment of the skin in the context of dermatological treatment. Accordingly, the formulations of the invention are desirably formulated as a cream, lotion, ointment, soap or body wash, shampoo, or a mask. However, the formulations can also be employed in make-up products, such as bases, blushes, lipsticks, and eye shadows, among others. They preferably include 0.001% by weight to 5% by weight, preferably 0.01% by weight to 2% by weight, oxidatively transformed carotenoid or 2-methyl-6-oxo-2,4-heptadienal.

### Antioxidants

Although the oxidatively transformed carotenoid and 2-methyl-6-oxo-2,4-heptadienal are products that result from the oxidation of carotenoids under certain conditions, these materials are themselves susceptible to further oxidation under ambient conditions. To prevent further oxidation it is desirable that the formulations of the invention contain one or more antioxidants and/or that the compositions be packaged under an atmosphere purged of oxygen gas. Antioxidants that can be used in combination with the oxidatively transformed carotenoid or 2-methyl-6-oxo-2,4-heptadienal can be selected from thiols (e.g., aurothioglucose, dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, and penta-, hexa- and heptathionine-sulphoximine), metal chelators (e.g, α-hydroxy-fatty acids, palmitic acid, phytic acid, lactoferrin, citric acid, lactic acid, and malic acid, humic acid, bile acid, bile extracts, bilirubin, biliverdin, EDTA, EGTA, and DTPA), vitamins (e.g., vitamin E, vitamin C, ascorbyl palmitate, Mg ascorbyl phosphate, and ascorbyl acetate), phenols (e.g., butylhydroxytoluene, butylhydroxyanisole, ubiquinol, nordihydroguaiaretic acid, trihydroxybutyrophenone), benzoates (e.g., coniferyl benzoate), uric acid, mannose, propyl gallate, selenium (e.g., selenium-methionine), stilbenes (e.g., stilbene oxide and trans-stilbene oxide), and combinations thereof.

Antioxidants that may be incorporated into the formulations of the invention include natural antioxidants prepared from plant extracts, such as extracts from aloe vera; avocado; chamomile; echinacea; ginko biloba; ginseng; green tea; heather; jojoba; lavender; lemon grass; licorice; mallow; oats; peppermint; St. John's wort; willow; wintergreen; wheat wild yam extract; marine extracts; and mixtures thereof.

The total amount of antioxidant included in the formulations can be from 0.001% to 3% by weight, preferably 0.01% to 1% by weight, in particular 0.05% to 0.5% by weight, based on the total weight of the formulation.

### Solubilizing Excipients

Oxidatively transfonned carotenoids have poor solubility in water at physiological pH. Accordingly, one or more solubilizing excipients may be a necessary component in the formulations of the invention.

Solubilization is taken to mean an improvement in the solubility by virtue of surface-active compounds that can convert substances that are insoluble or virtually insoluble in water into clear, or opalescent, aqueous solutions without changing the chemical structure of these substances in the process.

The solubilizates formed are notable for the fact that the substance is present in dissolved form in the molecular associations, micelles, of the surface-active compounds, which form in aqueous solution. The resulting solutions appear optically clear to opalescent.

Solubilizing excipients that may be used in the formulations of the invention include, without limitation, compounds belonging to the following classes: polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, ionic surfactants, tocopherol esters, and sterol esters. Commercially available examples for each class of excipient are provided below.

Polyethoxylated fatty acids may be used as excipients for the formulation of oxidatively transformed carotenoids. Examples of commercially available polyethoxylated fatty acid monoester surfactants include: PEG 4-100 monolaurate (Crodet L series, Croda), PEG 4-100 monooleate (Crodet O series, Croda), PEG 4-100 monostearate (Crodet S series, Croda, and Myrj Series, Atlas/ICI), PEG 400 distearate (Cithrol 4DS series, Croda), PEG 100, 200, or 300 monolaurate (Cithrol ML series, Croda), PEG 100, 200, or 300 monooleate (Cithrol MO series, Croda), PEG 400 dioleate (Cithrol 4DO series, Croda), PEG 400-1000 monostearate (Cithrol MS series, Croda), PEG-1 stearate (Nikkol MYS-1EX, Nikko, and Coster K1, Condea), PEG-2 stearate (Nikkol MYS-2, Nikko), PEG-2 oleate (Nikkol MYO-2, Nikko), PEG-4 laurate (Mapeg® 200 ML, PPG), PEG-4 oleate (Mapeg® 200 MO, PPG), PEG-4 stearate (Kessco® PEG 200 MS, Stepan), PEG-5 stearate (Nikkol TMGS-5, Nikko), PEG-5 oleate (Nikkol TMGO-5, Nikko), PEG-6 oleate (Algon OL 60, Auschem SpA), PEG-7 oleate (Algon OL 70, Auschem SpA), PEG-6 laurate (Kessco® PEG300 ML, Stepan), PEG-7 laurate (Lauridac 7, Condea), PEG-6 stearate (Kessco® PEG300 MS, Stepan), PEG-8 laurate (Mapeg® 400 ML, PPG), PEG-8 oleate (Mapeg® 400 MO, PPG), PEG-8 stearate (Mapeg® 400 MS, PPG), PEG-9 oleate (Emulgante A9, Condea), PEG-9 stearate (Cremophor S9, BASF), PEG-10 laurate (Nikkol MYL-10, Nikko), PEG-10 oleate (Nikkol MYO-10, Nikko), PEG-12 stearate (Nikkol MYS-10, Nikko), PEG-12 laurate (Kessco® PEG 600 ML, Stepan), PEG-12 oleate (Kessco® PEG 600 MO, Stepan), PEG-12 ricinoleate (CAS #9004-97-1), PEG-12 stearate (Mapeg® 600 MS, PPG), PEG-15 stearate (Nikkol TMGS-15, Nikko), PEG-15 oleate (Nikkol TMGO-15, Nikko), PEG-20 laurate (Kessco® PEG 1000 ML, Stepan), PEG-20 oleate (Kessco® PEG 1000 MO, Stepan), PEG-20 stearate (Mapeg® 1000 MS, PPG), PEG-25 stearate (Nikkol MYS-25, Nikko), PEG-32 laurate (Kessco® PEG 1540 ML, Stepan), PEG-32 oleate (Kessco® PEG 1540 MO, Stepan), PEG-32 stearate (Kessco® PEG 1540 MS, Stepan), PEG-30 stearate (Myrj 51), PEG-40 laurate (Crodet L40, Croda), PEG-40 oleate (Crodet O40, Croda), PEG-40 stearate (Emerest® 2715, Henkel), PEG-45 stearate (Nikkol MYS-45, Nikko), PEG-50 stearate (Myrj 53), PEG-55 stearate (Nikkol MYS-55, Nikko), PEG-100 oleate (Crodet O-100, Croda), PEG-100 stearate (Ariacel 165, ICI), PEG-200 oleate (Albunol 200 MO, Taiwan Surf.), PEG-400 oleate (LACTOMUL, Henkel), and PEG-600 oleate (Albunol 600 MO, Taiwan Surf). Formulations of the invention may include one or more of the polyethoxylated fatty acids above.

Polyethylene glycol fatty acid diesters may be used as excipients for the formulation of oxidatively transformed carotenoids. Examples of commercially available polyethylene glycol fatty acid diesters include: PEG-4 dilaurate (Mapeg® 200 DL, PPG), PEG-4 dioleate (Mapeg® 200 DO, PPG), PEG-4 distearate (Kesscog 200 DS, Stepan), PEG-6 dilaurate (Kessco® PEG 300 DL, Stepan), PEG-6 dioleate (Kessco® PEG 300 DO, Stepan), PEG-6 distearate (Kessco® PEG 300 DS, Stepan), PEG-8 dilaurate (Mapeg® 400 DL, PPG), PEG-8 dioleate (Mapeg® 400 DO, PPG), PEG-8 distearate (Mapeg® 400 DS, PPG), PEG-10 dipalmitate (Polyaldo 2PKFG), PEG-12 dilaurate (Kessco® PEG 600 DL, Stepan), PEG-12 distearate (Kessco® PEG 600 DS, Stepan), PEG-12 dioleate (Mapeg® 600 DO, PPG), PEG-20 dilaurate (Kessco® PEG 1000 DL, Stepan), PEG-20 dioleate (Kessco® PEG 1000 DO, Stepan), PEG-20 distearate (Kessco® PEG 1000 DS, Stepan), PEG-32 dilaurate (Kessco® PEG 1540 DL, Stepan), PEG-32 dioleate (Kessco® PEG 1540 DO, Stepan), PEG-32 distearate (Kessco® PEG 1540 DS, Stepan), PEG-400 dioleate (Cithrol 4DO series, Croda), and PEG-400 distearate Cithrol 4DS series, Croda). Formulations of the invention may include one or more of the polyethylene glycol fatty acid diesters above.

PEG-fatty acid mono- and di-ester mixtures may be used as excipients for the formulation of oxidatively transformed carotenoids. Examples of commercially available PEG-fatty acid mono- and di-ester mixtures include: PEG 4-150 mono, dilaurate (Kesscog PEG 200-6000 mono, Dilaurate, Stepan), PEG 4-150 mono, dioleate (Kessco® PEG 200-6000 mono, Dioleate, Stepan), and PEG 4-150 mono, distearate (Kessco® 200-6000 mono, Distearate, Stepan). Formulations of the invention may include one or more of the PEG-fatty acid mono- and di-ester mixtures above.

Polyethylene glycol glycerol fatty acid esters may be used as excipients for the formulation of oxidatively transformed carotenoids. Examples of commercially available polyethylene glycol glycerol fatty acid esters include: PEG-20 glyceryl laurate (Tagat® L, Goldschmidt), PEG-30 glyceryl laurate (Tagatg L2, Goldschmidt), PEG-15 glyceryl laurate (Glycerox L series, Croda), PEG-40 glyceryl laurate (Glycerox L series, Croda), PEG-20 glyceryl stearate (Capmul® EMG, ABITEC), and Aldog MS-20 KFG, Lonza), PEG-20 glyceryl oleate (Tagat® O, Goldschmidt), and PEG-30 glyceryl oleate (Tagat® O2, Goldschmidt). Formulations of the invention may include one or more of the polyethylene glycol glycerol fatty acid esters above.

Alcohol-oil transesterification products may be used as excipients for the formulation of oxidatively transformed carotenoids. Examples of commercially available alcohol-oil transesterification products include: PEG-3 castor oil (Nikkol CO-3, Nikko), PEG-5, 9, and 16 castor oil (ACCONON CA series, ABITEC), PEG-20 castor oil, (Emalex C-20, Nihon Emulsion), PEG-23 castor oil (Emulgante EL23), PEG-30 castor oil (Incrocas 30, Croda), PEG-35 castor oil (Incrocas-35, Croda), PEG-38 castor oil (Emulgante EL 65, Condea), PEG-40 castor oil (Emalex C-40, Nihon Emulsion), PEG-50 castor oil (Emalex C-50, Nihon Emulsion), PEG-56 castor oil (Eumulgin® PRT 56, Pulcra SA), PEG-60 castor oil (Nikkol CO-60TX, Nikko), PEG-100 castor oil, PEG-200 castor oil (Eumulging PRT 200, Pulcra SA), PEG-5 hydrogenated castor oil (Nikkol HCO-5, Nikko), PEG-7 hydrogenated castor oil (Cremophor WO7, BASF), PEG-10 hydrogenated castor oil (Nikkol HCO-10, Nikko), PEG-20 hydrogenated castor oil (Nikkol HCO-20, Nikko), PEG-25 hydrogenated castor oil (Simulsol® 1292, Seppic), PEG-30 hydrogenated castor oil (Nikkol HCO-30, Nikko), PEG-40 hydrogenated castor oil (Cremophor RH 40, BASF), PEG-45 hydrogenated castor oil (Cerex ELS 450, Auschem Spa), PEG-50 hydrogenated castor oil (Emalex HC-50, Nihon Emulsion), PEG-60 hydrogenated castor oil (Nikkol HCO-60, Nikko), PEG-80 hydrogenated castor oil (Nikkol HCO-80, Nikko), PEG-100 hydrogenated castor oil (Nikkol HCO-100, Nikko), PEG-6 corn oil (Labrafil® M 2125 CS, Gattefosse), PEG-6 almond oil (Labrafil® M 1966 CS, Gattefosse), PEG-6 apricot kernel oil (Labrafil® M 1944 CS, Gattefosse), PEG-6 olive oil (Labrafil® M 1980 CS, Gattefosse), PEG-6 peanut oil (Labrafil® M 1969 CS, Gattefosse), PEG-6 hydrogenated palm kernel oil (Labrafil® M 2130 BS, Gattefosse), PEG-6 palm kernel oil (Labrafil® M 2130 CS, Gattefosse), PEG-6 triolein (Labrafil® M 2735 CS, Gattefosse), PEG-8 corn oil (Labrafil® WL 2609 BS, Gattefosse), PEG-20 corn glycerides (Crovol M40, Croda), PEG-20 almond glycerides (Crovol A40, Croda), PEG-25 trioleate (TAGAT® TO, Goldschmidt), PEG-40 palm kernel oil (Crovol PK-70), PEG-60 corn glycerides (Crovol M70, Croda), PEG-60 almond glycerides (Crovol A70, Croda), PEG-4 caprylic/capric triglyceride (Labrafac® Hydro, Gattefosse), PEG-8 caprylic/capric glycerides (Labrasol, Gattefosse), PEG-6 caprylic/capric glycerides (SOFTIGEN®767, Huls), lauroyl macrogol-32 glyceride (GELUCIRE 44/14, Gattefosse), stearoyl macrogol glyceride (GELUCIRE 50/13, Gattefosse), mono, di, tri, tetra esters of vegetable oils and sorbitol (SorbitoGlyceride, Gattefosse), pentaerythrityl tetraisostearate (Crodamol PTIS, Croda), pentaerythrityl distearate (Albunol DS, Taiwan Surf.), pentaerythrityl tetraoleate (Liponate PO-4, Lipo Chem.), pentaerythrityl tetrastearate (Liponate PS-4, Lipo Chem.), pentaerythrityl tetracaprylate tetracaprate (Liponate PE-810, Lipo Chem.), and pentaerythrityl tetraoctanoate (Nikkol Pentarate 408, Nikko). Also included as oils in this category of surfactants are oil-soluble vitamins, such as vitamins A, D, E, K, etc. Thus, derivatives of these vitamins, such as tocopheryl PEG-1000 succinate (TPGS, available from Eastman), are also suitable surfactants. Formulations of the invention may include one or more of the alcohol-oil transesterification products above.

Polyglycerized fatty acids may be used as excipients for the formulation of oxidatively transformed carotenoids. Examples of commercially available polyglycerized fatty acids include: polyglyceryl-2 stearate (Nikkol DGMS, Nikko), polyglyceryl-2 oleate (Nikkol DGMO, Nikko), polyglyceryl-2 isostearate (Nikkol DGMIS, Nikko), polyglyceryl-3 oleate (Caprol® 3GO, ABITEC), polyglyceryl-4 oleate (Nikkol Tetraglyn 1-O, Nikko), polyglyceryl-4 stearate (Nikkol Tetraglyn 1-S, Nikko), polyglyceryl-6 oleate (Drewpol 6-1-O, Stepan), polyglyceryl-10 laurate (Nikkol Decaglyn 1-L, Nikko), polyglyceryl-10 oleate (Nikkol Decaglyn 1-O, Nikko), polyglyceryl-10 stearate (Nikkol Decaglyn 1-S, Nikko), polyglyceryl-6 ricinoleate (Nikkol Hexaglyn PR-15, Nikko), polyglyceryl-10 linoleate (Nikkol Decaglyn 1-LN, Nikko), polyglyceryl-6 pentaoleate (Nikkol Hexaglyn 5-O, Nikko), polyglyceryl-3 dioleate (Cremophor GO32, BASF), polyglyceryl-3 distearate (Cremophor GS32, BASF), polyglyceryl-4 pentaoleate (Nikkol Tetraglyn 5-O, Nikko), polyglyceryl-6 dioleate (Caprol® 6G20, ABITEC), polyglyceryl-2 dioleate (Nikkol DGDO, Nikko), polyglyceryl-10 trioleate (Nikkol Decaglyn 3-O, Nikko), polyglyceryl-10 pentaoleate (Nikkol Decaglyn 5-O, Nikko), polyglyceryl-10 septaoleate (Nikkol Decaglyn 7-O, Nikko), polyglyceryl-10 tetraoleate (Caprol® 10G40, ABITEC), polyglyceryl-10 decaisostearate (Nikkol Decaglyn 10-IS, Nikko), polyglyceryl-101 decaoleate (Drewpol 10-10-O, Stepan), polyglyceryl-10 mono, dioleate (Caprolg PGE 860, ABITEC), and polyglyceryl polyricinoleate (Polymuls, Henkel). Formulations of the invention may include one or more of the polyglycerized fatty acids above.

Propylene glycol fatty acid esters may be used as excipients for the formulation of oxidatively transformed carotenoids. Examples of commercially available propylene glycol fatty acid esters include: propylene glycol monocaprylate (Capryol 90, Gattefosse), propylene glycol monolaurate (Lauroglycol 90, Gattefosse), propylene glycol oleate (Lutrol OP2000, BASF), propylene glycol myristate (Mirpyl), propylene glycol monostearate (LIPO PGMS, Lipo Chem.), propylene glycol hydroxystearate, propylene glycol ricinoleate (PROPYMULS, Henkel), propylene glycol isostearate, propylene glycol monooleate (Myverol P-O6, Eastman), propylene glycol dicaprylate dicaprate (Captex® 200, ABITEC), propylene glycol dioctanoate (Captex® 800, ABITEC), propylene glycol caprylate caprate (LABRAFAC PG, Gattefosse), propylene glycol dilaurate, propylene glycol distearate (Kessco® PGDS, Stepan), propylene glycol dicaprylate (Nikkol Sefsol 228, Nikko), and propylene glycol dicaprate (Nikkol PDD, Nikko). Formulations the invention may include one or more of the propylene glycol fatty acid esters above.

Mixtures of propylene glycol esters and glycerol esters may be used as excipients for the formulation of oxidatively transformed carotenoids. One preferred mixture is composed of the oleic acid esters of propylene glycol and glycerol (Arlacel 186). Examples of these surfactants include: oleic (ATMOS 300, ARLACEL 186, ICI), stearic (ATMOS 150). Formulations of the invention may include one or more of the mixtures of propylene glycol esters and glycerol esters above.

Mono- and diglycerides may be used as excipients for the formulation of oxidatively transformed carotenoids. Examples of commercially available mono- and diglycerides include: monopalmitolein (C16:1) (Larodan), monoelaidin (C18:1) (Larodan), monocaproin (C6) (Larodan), monocaprylin (Larodan), monocaprin (Larodan), monolaurin (Larodan), glyceryl monomyristate (C14) (Nikkol MGM, Nikko), glyceryl monooleate (C18:1) (PECEOL, Gattefosse), glyceryl monooleate (Myverol, Eastman), glycerol monooleate/linoleate (OLICINE, Gattefosse), glycerol monolinoleate (Maisine, Gattefosse), glyceryl ricinoleate (Softigen® 701, Huls), glyceryl monolaurate (ALDO® MLD, Lonza), glycerol monopalmitate (Emalex GMS-P, Nihon), glycerol monostearate (Capmul® GMS, ABITEC), glyceryl mono- and dioleate (Capmul® GMO-K, ABITEC), glyceryl palmitic/stearic (CUTINA MD-A, ESTAGEL-G18), glyceryl acetate (Lamegin® EE, Grunau GmbH), glyceryl laurate (Imwitor® 312, Huls), glyceryl citrate/lactate/oleate/linoleate (Imwitor® 375, Huls), glyceryl caprylate (Imwitor® 308, Huls), glyceryl caprylate/caprate (Capmul® MCM, ABITEC), caprylic acid mono- and diglycerides (Imwitor® 988, Huls), caprylic/capric glycerides (Imwitor® 742, Huls), Mono-and diacetylated monoglycerides (Myvacet® 9-45, Eastman), glyceryl monostearate (Aldo® MS, Arlacel 129, ICI), lactic acid esters of mono and diglycerides (LAMEGIN GLP, Henkel), dicaproin (C6) (Larodan), dicaprin (C10) (Larodan), dioctanoin (C8) (Larodan), dimyristin (C14) (Larodan), dipalmitin (C16) (Larodan), distearin (Larodan), glyceryl dilaurate (C12) (Capmulg GDL, ABITEC), glyceryl dioleate (Capmul® GDO, ABITEC), glycerol esters of fatty acids (GELUCIRE 39/01, Gattefosse), dipalmitolein (C16:1) (Larodan), 1,2 and 1,3-diolein (C18:1) (Larodan), dielaidin (C18:1) (Larodan), and dilinolein (C18:2) (Larodan). Formulations of the invention may include one or more of the mono- and diglycerides above.

Sterol and sterol derivatives may be used as excipients for the formulation of oxidatively transformed carotenoids. Examples of commercially available sterol and sterol derivatives include: cholesterol, sitosterol, lanosterol, PEG-24 cholesterol ether (Solulan C-24, Amerchol), PEG-30 cholestanol (Phytosterol GENEROL series, Henkel), PEG-25 phytosterol (Nikkol BPSH-25, Nikko), PEG-5 soyasterol (Nikkol BPS-5, Nikko), PEG-10 soyasterol (Nikkol BPS-10, Nikko), PEG-20 soyasterol (Nikkol BPS-20, Nikko), and PEG-30 soyasterol (Nikkol BPS-30, Nikko). Formulations of the invention may include one or more of the sterol and sterol derivatives above.

Polyethylene glycol sorbitan fatty acid esters may be used as excipients for the formulation of oxidatively transformed carotenoids. Examples of commercially available polyethylene glycol sorbitan fatty acid esters include: PEG-10 sorbitan laurate (Liposorb L-10, Lipo Chem.), PEG-20 sorbitan monolaurate (Tween® 20, Atlas/ICI), PEG-4 sorbitan monolaurate (Tween® 21, Atlas/ICI), PEG-80 sorbitan monolaurate (Hodag PSML-80, Calgene), PEG-6 sorbitan monolaurate (Nikkol GL-1, Nikko), PEG-20 sorbitan monopalmitate (Tween® 40, Atlas/ICI), PEG-20 sorbitan monostearate (Tween® 60, Atlas/ICI), PEG-4 sorbitan monostearate (Tween® 61, Atlas/ICI), PEG-8 sorbitan monostearate (DACOL MSS, Condea), PEG-6 sorbitan monostearate (Nikkol TS106, Nikko), PEG-20 sorbitan tristearate (Tween® 65, Atlas/ICI), PEG-6 sorbitan tetrastearate (Nikkol GS-6, Nikko), PEG-60 sorbitan tetrastearate (Nikkol GS-460, Nikko), PEG-5 sorbitan monooleate (Tween® 81, Atlas/ICI), PEG-6 sorbitan monooleate (Nikkol TO-106, Nikko), PEG-20 sorbitan monooleate (Tween® 80, Atlas/ICI), PEG-40 sorbitan oleate (Emalex ET 8040, Nihon Emulsion), PEG-20 sorbitan trioleate (Tween® 85, Atlas/ICI), PEG-6 sorbitan tetraoleate (Nikkol GO-4, Nikko), PEG-30 sorbitan tetraoleate (Nikkol GO-430, Nikko), PEG-40 sorbitan tetraoleate (Nikkol GO-440, Nikko), PEG-20 sorbitan monoisostearate (Tween® 120, Atlas/ICI), PEG sorbitol hexaoleate (Atlas G-1086, ICI), polysorbate 80 (Tween® 80, Pharma), polysorbate 85 (Tween® 85, Pharma), polysorbate 20 (Tween® 20, Pharma), polysorbate 40 (Tween® 40, Pharma), polysorbate 60 (Tween® 60, Pharma), and PEG-6 sorbitol hexastearate (Nikkol GS-6, Nikko). Formulations of the invention may include one or more of the polyethylene glycol sorbitan fatty acid esters above.

Polyethylene glycol alkyl ethers may be used as excipients for the formulation of oxidatively transformed carotenoids. Examples of commercially available polyethylene glycol alkyl ethers include: PEG-2 oleyl ether, oleth-2 (Brij 92/93, Atlas/ICI), PEG-3 oleyl ether, oleth-3 (Volpo 3, Croda), PEG-5 oleyl ether, oleth-5 (Volpo 5, Croda), PEG-10 oleyl ether, oleth-10 (Volpo 10, Croda), PEG-20 oleyl ether, oleth-20 (Volpo 20, Croda), PEG-4 lauryl ether, laureth-4 (Brij 30, Atlas/ICI), PEG-9 lauryl ether, PEG-23 lauryl ether, laureth-23 (Brij 35, Atlas/ICI), PEG-2 cetyl ether (Brij 52, ICI), PEG-10 cetyl ether (Brij 56, ICI), PEG-20 cetyl ether (BriJ 58, ICI), PEG-2 stearyl ether (Brij 72, ICI), PEG-10 stearyl ether (Brij 76, ICI), PEG-20 stearyl ether (Brij 78, ICI), and PEG-100 stearyl ether (Brij 700, ICI). Formulations of the invention may include one or more of the polyethylene glycol alkyl ethers above.

Sugar esters may be used as excipients for the formulation of oxidatively transformed carotenoids. Examples of commercially available sugar esters include: sucrose distearate (SUCRO ESTER 7, Gattefosse), sucrose distearate/monostearate (SUCRO ESTER 11, Gattefosse), sucrose dipalmitate, sucrose monostearate (Crodesta F-160, Croda), sucrose monopalmitate (SUCRO ESTER 15, Gattefosse), and sucrose monolaurate (Saccharose monolaurate 1695, Mitsubisbi-Kasei). Formulations of the invention may include one or more of the sugar esters above.

Polyethylene glycol alkyl phenols may be used as excipients for the formulation of oxidatively transformed carotenoids. Examples of commercially available polyethylene glycol alkyl phenols include: PEG-10-100 nonylphenol series (Triton X series, Rohm & Haas) and PEG-15-100 octylphenol ether series (Triton N-series, Rohm & Haas). Formulations of the invention may include one or more of the polyethylene glycol alkyl phenols above.

Polyoxyethylene-polyoxypropylene block copolymers may be used as excipients for the formulation of oxidatively transformed carotenoids. These surfactants are available under various trade names, including one or more of Synperonic PE series (ICI), Pluronic® series (BASF), Lutrol (BASF), Supronic, Monolan, Pluracare, and Plurodac. The generic term for these polymers is “poloxamer” (CAS 9003-11-6). These polymers have the formula I:

HO(C₂H₄O)ₐ(C₃H₆O)_{b}(C₂H₄O)ₐH I

where “a” and “b” denote the number of polyoxyethylene and polyoxypropylene units, respectively. Formulations of the invention may include one or more of the polyoxyethylene-polyoxypropylene block copolymers above.

Polyoxyethylenes, such as PEG 300, PEG 400, and PEG 600, may be used as excipients for the formulation of oxidatively transformed carotenoids.

Sorbitan fatty acid esters may be used as excipients for the formulation of oxidatively transformed carotenoids. Examples of commercially sorbitan fatty acid esters include: sorbitan monolaurate (Span-20, Atlas/ICI), sorbitan monopalmitate (Span-40, Atlas/ICI), sorbitan monooleate (Span-80, Atlas/ICI), sorbitan monostearate (Span-60, Atlas/ICI), sorbitan trioleate (Span-85, Atlas/ICI), sorbitan sesquioleate (Arlacel-C, ICI), sorbitan tristearate (Span-65, Atlas/ICI), sorbitan monoisostearate (Crill 6, Croda), and sorbitan sesquistearate (Nikkol SS-15, Nikko). Formulations of the invention may include one or more of the sorbitan fatty acid esters above.

Esters of lower alcohols (C2 to C4) and fatty acids (C8 to C18) are suitable surfactants for use in the invention. Examples of these surfactants include: ethyl oleate (Crodamol EO, Croda), isopropyl myristate (Crodamol IPM, Croda), isopropyl palmitate (Crodamol IPP, Croda), ethyl linoleate (Nikkol VF-E, Nikko), and isopropyl linoleate (Nikkol VF-IP, Nikko). Formulations of the invention may include one or more of the lower alcohol fatty acid esters above.

Ionic surfactants may be used as excipients for the formulation of oxidatively transformed carotenoids. Examples of useful ionic surfactants include: sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate (dodecyl), sodium tetradecyl sulfate, sodium lauryl sarcosinate, sodium dioctyl sulfosuccinate, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco cheno deoxycholate, sodium cholylsarcosinate, sodium N-methyl taurocholate, egg yolk phosphatides, hydrogenated soy lecithin, dimyristoyl lecithin, lecithin, hydroxylated lecithin, lysophosphatidylcholine, cardiolipin, sphingomyelin, phosphatidylcholine, phosphatidyl ethanolamine, phosphatidic acid, phosphatidyl glycerol, phosphatidyl serine, diethanolamine, phospholipids, polyoxyethylene-10 oleyl ether phosphate, esterification products of fatty alcohols or fatty alcohol ethoxylates, with phosphoric acid or anhydride, ether carboxylates (by oxidation of terminal OH group of, fatty alcohol ethoxylates), succinylated monoglycerides, sodium stearyl fumarate, stearoyl propylene glycol hydrogen succinate, mono/diacetylated tartaric acid esters of mono- and diglycerides, citric acid esters of mono-, diglycerides, glyceryl-lacto esters of fatty acids, acyl lactylates, lactylic esters of fatty acids, sodium stearoyl-2-lactylate, sodium stearoyl lactylate, alginate salts, propylene glycol alginate, ethoxylated alkyl sulfates, alkyl benzene sulfones, α-olefin sulfonates, acyl isethionates, acyl taurates, alkyl glyceryl ether sulfonates, sodium octyl sulfosuccinate, sodium undecylenamideo-MEA-sulfosuccinate, hexadecyl triammonium bromide, decyl trimethyl ammonium bromide, cetyl trimethyl ammonium bromide, dodecyl ammonium chloride, alkyl benzyldimethylammonium salts, diisobutyl phenoxyethoxydimethyl benzylammonium salts, alkylpyridinium salts, betaines (trialkylglycine), lauryl betaine (N-lauryl,N,N-dimethylglycine), and ethoxylated amines (polyoxyethylene-15 coconut amine). For simplicity, typical counterions are provided above. It will be appreciated by one skilled in the art, however, that any bioacceptable counterion may be used. For example, although the fatty acids are shown as sodium salts, other cation counterions can also be used, such as, for example, alkali metal cations or ammonium. Formulations of the invention may include one or more of the ionic surfactants above.

Tocopherol esters and sterol esters, as described in U.S. Pat. Nos. 6,632,443 and 6,191,172, each of which is incorporated herein by reference, may be used as excipients for the formulation of oxidatively transformed carotenoids. These tocopherol and sterol esters are described by formula II:

{X-OOC—[(CH₂)ₙ—COO]ₘ}ₚ-Y II

wherein X is selected from α-tocopherol, β-tocopherol, γ-tocopherol, δ-tocopherol, cholesterol, 7-dehydrocholesterol, campesterol, sitosterol, ergosterol, and stigmasterol; p is 1 or 2; m is 0 or 1; n is an integer from 0 to 18; and Y is a hydrophilic moiety selected from polyalcohols, polyethers, and derivatives thereof.

The solubilizing excipients present in the formulations of the invention are present in amounts such that the carrier forms a clear, or opalescent, aqueous dispersion of oxidatively transformed carotenoid or 2-methyl-6-oxo-2,4-heptadienal. The relative amounts of surfactants required are readily determined by observing the solubility properties of the resultant oxidatively transformed carotenoid dispersion, as determined using standard techniques for measuring solubilities. The optical clarity of the aqueous dispersion can be measured using standard quantitative techniques for turbidity assessment. For example, a formulation of the invention can include from 0.001% to 10% by weight, preferably 0.01% to 5% by weight, solubilizing excipient.

All uses of solubilizing excipients described herein are also applicable to fonnulations of 2-methyl-6-oxo-2,4-heptadienal.

### Other Active Ingredients

The formulations of the invention can be used in combination with any second active ingredient described herein. Desirably, the oxidatively transformed carotenoid or 2-methyl-6-oxo-2,4-heptadienal and the second active ingredient are formulated together. The amount of a second active ingredient included will depend on the desired effect and the active ingredient that is selected. In general, the amount of a second active ingredient varies from about 0.0001% to about 20%, preferably from about 0.01% to about 10%, or even about 0.1% to about 5% by weight.

### Uses

The formulations of the invention can be used to treat skin conditions, such as dandruff, psoriasis, eczema, keloids, keratosis, and warts. Furthermore, the formulations can be used to treat the symptoms of photoaging of the skin, such as coarseness, wrinkles, mottled pigmentation, and sallowness.

The application regimen (i.e., daily, weekly, etc.) will primarily depend upon the longevity (e.g., metabolism, half-life in the skin) of the agents and the skin condition to be treated. For topical administration, the regimen may also be affected by bathing, perspiration, and the extent of sunlight exposure. Usually, the formulation will be administered at least once daily.

The weight concentration of oxidatively transformed carotenoid or 2-methyl-6-oxo-2,4-heptadienal in the formulation will usually be 0.0001% to 5%, more usually 0.001% to 3%. Normally, about 1 to 50 mg of formulation will be applied per cm² of skin. Desirably, the oxidatively transformed carotenoid or 2-methyl-6-oxo-2,4-heptadienal are formulated with other active ingredients as described below.

### Photoaging

The oxidatively transformed carotenoid or 2-methyl-6-oxo-2,4-heptadienal can be formulated as a cream, lotion, or spray and applied to the skin to prevent and treat photoaging. To treat photoaging, the oxidatively transformed carotenoid or 2-methyl-6-oxo-2,4-heptadienal is desirably applied once or twice daily.

The oxidatively transformed carotenoid or 2-methyl-6-oxo-2,4-heptadienal may be formulated with anti-wrinkle and/or anti-aging agents, such as hydroxy acids. Hydroxy acids include, without limitation, C₂-C₃₀ alpha-hydroxy acids such as glycolic acid, lactic acid, 2-hydroxy butanoic acid, malic acid, citric acid tartaric acid, alpha-hydroxyethanoic acid, and hydroxycaprylic acid; beta hydroxy acids, such as salicylic acid; and polyhydroxy acids, such as gluconolactone (G4); and mixtures of thereof. Further anti-wrinkle agents include retinoic acid, gamma-linolenic acid; and mixtures thereof. Skin peel agents for example phenol, phytic acid and acetic acid may also be used.

Furthermore, the oxidatively transformed carotenoid or 2-methyl-6-oxo-2,4-heptadienal can be formulated in combination with a photoprotective ingredient as a lotion, cream, or spray and applied to the skin as a sunscreen. The sunscreens may be applied prior to exposure to the sun and as needed thereafter.

Any photoprotective ingredient offering protection against ultraviolet radiation by absorbing, scattering or reflecting the ultraviolet radiation may be used herein. The sunprotection factor (SPF) in the final formulation varies between 2 and 30, although products with SPFs up to 100 may be formulated. Photoprotective ingredients which may be included in the formulation of a sunscreen include amino benzoic acids, such as para-amino benzoic acid (PABA), glyceryl-PABA (Lisadimate), Padimate O, or Roxadimate; anthrinalates, including methylanthrynilate; benzophenones, including dioxybenzone, oxybenzone and sulisobenzone; camphor derivatives, including 3-(4-methylbenzylidene) camphor, 3-benzylidene camphor; cinnamates including DEA-p-methoxycinnamate, ethyl-hexyl p-methoxy cinnamate, octocrylene, octyl methoxy cinnamate (Parasol MCX); dibenzoyl methanes, including butylmethoxydibenzoylmethane (Parsol 1789); salicylates, including homomenthyl salicylate, octyl salicylate, trolamine methyl salicylate; metal oxides, including titanium dioxide, zinc oxide and iron oxide; 2-phenylbenzimidazole-5-sulfonic acid; 4,4-methoxy-t-butyldibenzoylmethane; and mixtures thereof. Further non-limiting examples of active ingredients which may be included in the formulation of a sunscreen are described in U.S. Pat. Nos. 5,087,445; 5,073,372; and 5,160,731; each of which are incorporated herein by reference.

The sunscreens can also include ingredients that provide a sunless tan, such as dihydroxyacetone (DHA); glyceryl aldehyde; tyrosine and tyrosine derivatives such as malyltyrosine, tyrosine glucosinate, and ethyl tyrosine; phospho-DOPA, indoles and derivatives; and mixtures thereof.

### Psoriasis

The oxidatively transformed carotenoid or 2-methyl-6-oxo-2,4-heptadienal can be formulated as a cream, lotion, or spray and applied to the skin to treat psoriasis. The formulation is applied directly to the diseased area of skin once or twice daily.

When used to treat psoriasis, the formulations can further include one or more additional anti-psoriasis agents selected from salicylic acid; corticosteroids; 5-fluorouracil; epinephrine; anthralin; vitamin D3 analogs, such as calcipotriene; methotrexate; masprocol; trimethaxate gluconate; retinoids; cyclosporin; paclitaxel; 5-amino levulinic acid; bergasol; benzoporphyrins; antibodies, such as ABX-IL8 antibody, CD11a monoclonal antibody and ICM3 monoclonal antibody; enzyme inhibitors, including tryptase inhibitor and phospholipase A-2 inhibitors; angiogenesis blocking agents; T-cell blocking agents and mixtures thereof.

### Dandruff

The oxidatively transformed carotenoid or 2-methyl-6-oxo-2,4-heptadienal can be formulated as a shampoo and applied to the scalp for the treatment of dandruff. The formulation is applied to the hair and scalp daily.

The oxidatively transformed carotenoid or 2-methyl-6-oxo-2,4-heptadienal may be formulated with other anti-dandruff agents, such as chloroxine, coal tar, ketoconazole, pyrithione, salicylic acid, zinc salts, selenium sulfide, piroctone olamine, and sulphur, among others.

### Eczema

The oxidatively transformed carotenoid or 2-methyl-6-oxo-2,4-heptadienal can be formulated as a cream, lotion, or spray and applied to the skin to treat eczema. The formulation is applied directly to the diseased area of skin once or twice daily.

The oxidatively transformed carotenoid or 2-methyl-6-oxo-2,4-heptadienal may be formulated with other anti-eczema agents, such as an antihistamine or a corticosteroid.

For use in any of the compositions or methods described herein, suitable antihistamines include, without limitation, mepyramine, brompheniramine, chlorpheniramine, dimethindene, acrivastine, pheniramine, triprolidine, buclizine, cyclizine, hydroxyzine, meclizine, oxatomide, cetirizine, levocetirizine, azatadine, cyproheptadine, diphenylpyraline, ketotifen, desloratadine, ebastine, fexofenadine, levocabastine, loratadine, mizolastine, olopatadine, carbinoxamine, clemastine, dimenhydrinate, diphenhydramine, doxylamine, phenyltoloxamine, antazoline, pyrilamine, tripelennamine, methdilazine, promethazine, azelastine, emedastine, and epinastine.

For use in any of the compositions or methods described herein, suitable corticosteroids include, without limitation, alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, and triamcinolone acetonide.

### Warts, Keloids, and Keratosis

The oxidatively transformed carotenoid or 2-methyl-6-oxo-2,4-heptadienal can be formulated as a cream, lotion, or spray and applied to the skin to treat warts, keloids, or keratosis. For these uses the formulation can be applied directly to the affected area once or twice daily.

The oxidatively transformed carotenoid or 2-methyl-6-oxo-2,4-heptadienal may be formulated with other anti-wart agents, such as diphencyprone, aldara, imiquimod, corticosteroids, or salicylic acid; other anti-keloid agents, such as corticosteroids; and other anti-keratosis agents, such as 5-fluoro uracil or imiquod, as needed.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLE 1

### Anti-Eczema Cream

| Component | Wt % |
|---|---|
| Paraffin oil | 10.00 |
| Petrolatum | 4.00 |
| Wool wax alcohol | 1.00 |
| PEG 7-hydrogenated castor oil | 3.00 |
| Aluminium stearate | 0.40 |
| Propyl gallate | 0.10 |
| Glycerol | 2.00 |
| Oxidatively transformed | 3.00 |
| carotenoid | |
| Hydrocortisone | 2.50 |
| Loratadine | 0.20 |
| Water, preservative, | to 100.00 |
| and perfume | |

### EXAMPLE 2

### Sunscreen Lotion

| Component | Wt % |
|---|---|
| C₁₂₋₁₅ alkyl benzoate | 20.00 |
| Sorbitan oleate | 4.00 |
| Sorbitan stearate | 3.00 |
| Glyceryl stearate | 3.00 |
| Stearic acid | 2.00 |
| Hydrogenated vegetable oil | 2.00 |
| Vitamin E | 2.00 |
| Vitamin C palmitate | 0.20 |
| Methoxycinnamate | 0.20 |
| Oxidatively transformed carotenoid | 0.20 |
| Glycerol | 5.00 |
| Water, preservative, | to 100.00 |
| and perfume | |

### EXAMPLE 3

### Anti-Psoriasis Lotion

| Component | Wt % |
|---|---|
| Paraffin oil | 8.00 |
| Isopropyl palmitate | 3.00 |
| Petrolatum | 4.00 |
| Cetearyl alcohol | 2.00 |
| PEG 40-castor oil | 0.50 |
| Sodium cetearyl sulphate | 0.50 |
| Sodium carbomer | 0.40 |
| Prednisolone | 0.50 |
| Salicylic acid | 0.25 |
| Glycerol | 3.00 |
| Vitamin E | 0.20 |
| Oxidatively transformed carotenoid | 3.00 |
| Water, preservative | to 100.00 |
| and perfume | |

### EXAMPLE 4

### Anti-Dandruff Shampoo

| Component | Wt % |
|---|---|
| Ammonium Lauryl Sulfate | 7.00 |
| Ammonium Laureth Sulfate | 9.00 |
| Sodium Lauroamphoacetate | 5.00 |
| Malic Acid | 2.00 |
| Sodium Hydroxide | to pH 5.0 |
| Salicylic Acid | 2.00 |
| Pyrithione Zinc | 1.00 |
| Polyquaternium-10 | 0.50 |
| Ascorbyl acetate | 0.20 |
| 2-methyl-6-oxo-2,4-heptadienal | 0.50 |
| Water, preservative, | to 100.00 |
| dye, and perfume | |

### EXAMPLE 5

### Anti-Wart Cream

| Component | Wt % |
|---|---|
| Paraffin oil | 7.00 |
| Avocado oil | 4.00 |
| Glyceryl monostearate | 2.00 |
| Sodium stearate | 1.00 |
| Titanium dioxide | 1.00 |
| Sodium lactate | 3.00 |
| Glycerol | 3.00 |
| Vitamin E | 0.20 |
| 2-methyl-6-oxo-2,4-heptadienal | 3.00 |
| Imiquimod | 3.00 |
| Salicylic acid | 1.00 |
| Hydrocortisone | 2.50 |
| Water, preservative, | to 100.00 |
| and perfume | |

### EXAMPLE 6

### Lip care stick

| Component | Wt % |
|---|---|
| Hydrogenated castor oil | 4.00 |
| Ceresin | 8.00 |
| Beeswax | 4.00 |
| Carnauba wax | 2.00 |
| Petrolatum | 40.00 |
| Butylhydroxytoluene | 0.02 |
| Methoxycinnamate | 1.00 |
| 2-methyl-6-oxo-2,4-heptadienal | 0.30 |
| Paraffin oil, preservatives, | to 100.00 |
| and dyes | |

### EXAMPLE 7

### Liposome-containing Gel

| Component | Wt % |
|---|---|
| Lecithin | 6.00 |
| Shea butter | 3.00 |
| Oxidatively transformed carotenoid | 0.50 |
| Butylated hydroxyanisole | 0.20 |
| Sodium citrate | 0.50 |
| Glycine | 0.20 |
| Urea | 0.20 |
| Sodium PCA | 0.50 |
| Hydrolysed collagen | 2.00 |
| Xanthan gum | 1.40 |
| Sorbitol | 3.00 |
| Water, preservative, | to 100.00 |
| and perfume | |

### OTHER EMBODIMENTS

All publications and patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims.

What we claim is:

1. A composition formulated for topical administration comprising from 0.0001% to 5% (w/w) oxidatively transformed carotenoid.

2. The composition of claim 1, further comprising an antioxidant.

3. The composition of claim 2, wherein said antioxidant is selected from thiols, sulphoximines, metal chelators, fatty acids, vitamins, phenols, stilbenes, uric acid, mannose, selenium and propyl gallate.

4. The composition of claim 3, wherein said antioxidant is vitamin E.

5. The composition of claim 1, further comprising one or more solubilizing excipients wherein the class of excipient is selected from the group consisting of polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, ionic surfactants, tocopherol esters, and sterol esters.

6. The composition of claim 1, further comprising a UV light blocker, a corticosteroid, an antihistamine, a retinoid, 5-fluorouracil, epinephrine, anthralin, calcipotriene, methotrexate, masprocol, trimethaxate gluconate, cyclosporin, paclitaxel, 5-amino levulinic acid, bergasol, benzoporphyrin, hydroxy acid, retinoic acid, diphencyprone, aldara, imiquimod, gamma-linolenic acid, chloroxine, coal tar, ketoconazole, pyrithione, salicylic acid, zinc salts, selenium sulfide, piroctone olamine, sulphur, or mixtures thereof.

7. The composition of claim 6, wherein said UV light blocker is from a class selected from amino benzoic acids, benzophenones, camphors, cinnamates, dibenzoyl methanes, salicylates, metal oxides, and mixtures thereof.

8. The composition of claim 6, wherein said antihistamine is mepyramine, diphenhydramine, or antazoline.

9. The composition of claim 6, wherein said corticosteroid is alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, or triamcinolone acetonide.

10. The composition of claim 1, wherein said composition is formulated as a cream, lotion, spray, stick, ointment, soap, body wash, shampoo, or mask.

11. A composition formulated for topical administration comprising from 0.0001% to 5% (w/w) 2-methyl-6-oxo-2,4-heptadienal.

12. The composition of claim 11, further comprising an antioxidant.

13. The composition of claim 12, wherein said antioxidant is selected from thiols, sulphoximines, metal chelators, fatty acids, vitamins, phenols, stilbenes, uric acid, mannose, selenium and propyl gallate.

14. The composition of claim 13, wherein said antioxidant is vitamin E.

15. The composition of claim 11, further comprising one or more solubilizing excipients wherein the class of excipient is selected from the group consisting of polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, ionic surfactants, tocopherol esters, and sterol esters.

16. The composition of claim 11, further comprising a UV light blocker, a corticosteroid, an antihistamine, a retinoid, 5-fluorouracil, epinephrine, anthralin, calcipotriene, methotrexate, masprocol, trimethaxate gluconate, cyclosporin, paclitaxel, 5-amino levulinic acid, bergasol, benzoporphyrin, hydroxy acid, retinoic acid, diphencyprone, aldara, imiquimod, gamma-linolenic acid, chloroxine, coal tar, ketoconazole, pyrithione, salicylic acid, zinc salts, selenium sulfide, piroctone olamine, sulphur, or mixtures thereof.

17. The composition of claim 16, wherein said Uv light blocker is from a class selected from amino benzoic acids, benzophenones, camphors, cinnamates, dibenzoyl methanes, salicylates, metal oxides, and mixtures thereof.

18. The composition of claim 16, wherein said antihistamine is mepyramine, diphenhydramine, or antazoline.

19. The composition of claim 16, wherein said corticosteroid is alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, or triamcinolone acetonide.

20. The composition of claim 11, wherein said composition is formulated as a cream, lotion, spray, stick, ointment, soap, body wash, shampoo, or mask.

21. A method of treating a skin condition in a mammal, said method comprising the step of applying a composition of claim 1 to the skin of said mammal in an amount sufficient to treat said skin condition.

22. The method of claim 21, wherein said skin condition is dandruff.

23. The method of claim 22, further comprising the administration of chloroxine, coal tar, ketoconazole, pyrithione, salicylic acid, zinc salts, selenium sulfide, piroctone olamine, sulphur, or combination thereof to said mammal.

24. The method of claim 21, wherein said skin condition is psoriasis.

25. The method of claim 24, further comprising the administration of a corticosteroid, 5-fluorouracil, epinephrine, anthralin, calcipotriene, methotrexate, masprocol, trimethaxate gluconate, retinoids, cyclosporin, paclitaxel, 5-amino levulinic acid, bergasol, benzoporphyrin, or combinatinon thereof to said mammal.

26. The method of claim 21, wherein said skin condition is photoaging.

27. The method of claim 26, further comprising the administration of a UV light blocker, hydroxy acid, retinoic acid, ganima-linolenic acid, or combination thereof to said mammal.

28. The method of claim 21, wherein said skin condition is eczema.

29. The method of claim 28, further comprising the administration of an antihistamine, corticosteroid, or combination thereof to said mammal.

30. The method of claims 25 or 29, wherein said corticosteroid is alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, or triamcinolone acetonide.

31. The method of claim 29, wherein said antihistamine is mepyramine, diphenhydramine, or antazoline.

32. The method of claim 21, wherein said skin condition is selected from warts, keloids, and keratosis.

33. A method of treating a skin condition in a mammal, said method comprising the step of applying a composition of claim 11 to the skin of said mammal in an amount sufficient to treat said skin condition.

34. The method of claim 33, wherein said skin condition is dandruff.

35. The method of claim 34, further comprising the administration of chloroxine, coal tar, ketoconazole, pyrithione, salicylic acid, zinc salts, selenium sulfide, piroctone olamine, sulphur, or combination thereof to said mammal.

36. The method of claim 33, wherein said skin condition is psoriasis.

37. The method of claim 36, further comprising the administration of a corticosteroid, 5-fluorouracil, epinephrine, anthralin, calcipotriene, methotrexate, masprocol, trimethaxate gluconate, retinoids, cyclosporin, paclitaxel, 5-amino levulinic acid, bergasol, benzoporphyrin, or combinatinon thereof to said mammal.

38. The method of claim 33, wherein said skin condition is photoaging.

39. The method of claim 38, further comprising the administration of a UV light blocker, hydroxy acid, retinoic acid, gamma-linolenic acid, or combination thereof to said mammal.

40. The method of claim 33, wherein said skin condition is eczema.

41. The method of claim 40, further comprising the administration of an antihistamine, corticosteroid, or combination thereof to said mammal.

42. The method of claims 37 or 41, wherein said corticosteroid is alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, or triamcinolone acetonide.

43. The method of claim 41, wherein said antihistamine is mepyramine, diphenhydramine, or antazoline.

44. The method of claim 33, wherein said skin condition is selected from warts, keloids, and keratosis.

45. A container comprising an atmosphere purged of oxygen gas and a composition comprising from 0.0001% to 5% (w/w) oxidatively transformed carotenoid, wherein said composition formulated for topical administration.

46. A container comprising an atmosphere purged of oxygen gas and a composition comprising from 0.0001% to 5% (w/w) 2-methyl-6-oxo-2,4-heptadienal, wherein said composition formulated for topical administration.

47. A composition comprising 0.001% to 3% (w/w) antioxidant and oxidatively transformed carotenoid.

48. A composition comprising 0.001% to 3% (w/w) antioxidant and 2-methyl-6-oxo-2,4-heptadienal.
